SEQUENCE LISTING (10 sequences)

SEQ ID NO: 1 - protein, 640 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLDCEVYLCDTMNEKCKPTCSGTRFRSGSVIDQSRVLNLGPITRKGVQATVSRAFSSLGLLKVWLPLLLSATLTLTFQ

SEQ ID NO: 2 - protein, 587 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLDCEVYLCDTMNEKCKPTCSGTRF

SEQ ID NO: 3 - protein, 22 aa, Homo sapiens
SVIDQSRVLNLGPITRKGVQAT

SEQ ID NO: 4 - protein, 10 aa, Homo sapiens
VLNLGPITRK

SEQ ID NO: 5 - protein, 15 aa, Homo sapiens
DRDNRDWVSVVTPAR

SEQ ID NO: 6 - protein, 37 aa, Artificial Sequence
  Description of Artificial Sequence Synthetic polypeptide
DRDNRDWVSVVTPARSVIDQSRVLNLGPITRKGVQAT

SEQ ID NO: 7 - protein, 8 aa, Artificial Sequence
  Description of Artificial Sequence Synthetic peptide
GGGGGGGG

SEQ ID NO: 8 - protein, 5 aa, Artificial Sequence
  Description of Artificial Sequence Synthetic peptide
GGGGS

SEQ ID NO: 9 - protein, 12 aa, Artificial Sequence
  Description of Artificial Sequence Synthetic peptide
GSAGSAAGSGEF

SEQ ID NO: 10 - protein, 15 aa, Artificial Sequence
  Description of Artificial Sequence Synthetic peptide
  MOD_RES (8)..(8): Any amino acid
  MOD_RES (10)..(11): Any amino acid
DRDNRDWXSXXTPAR

(19) United States
(12) Patent Application Publication
(10) Pub. No.: US 2026/0177545 A1
(43) Pub. Date: Jun. 25, 2026

(54) MATERIALS AND METHODS FOR QUANTIFYING PRECURSOR TAMM-HORSFALL PROTEIN

(71) Applicants: The Trustees of Indiana University, Bloomington, IN (US); The United States of America as Represented by the Department of Veterans Affairs, Washington, DC (US)

(72) Inventors: Tarek M. Ashkar, Carmel, IN (US); Radmila Micanovic, Indianapolis, IN (US); Kaice LaFavers, Indianapolis, IN (US); Kavish Patidar, Westfield, IN (US)

(21) Appl. No.: 18/272,112

(22) PCT Filed: Jan. 14, 2022
(86) PCT No.: PCT/US2022/012457

§ 371 Date: Jul. 13, 2023

Related U.S. Application Data
(60) Provisional application No. 63/137,258, filed on Jan. 14, 2021.
(60) Provisional application No. 63/148,897, filed on Feb. 12, 2021.

(51) Int. Cl. G01N 33/543; C07K 14/47; C12N 15/62; C12N 15/63; G01N 33/68

(52) U.S. Cl. CPC G01N 33/54366; C07K 14/4703; C12N 15/62; C12N 15/63; G01N 33/6893; C07K 2319/00; G01N 2333/4704; G01N 2800/347; G01N 2800/50

(57) ABSTRACT

Embodiments herein provide compositions, methods, uses for detecting precursor Tamm-Horsfall protein (THP) in a biological sample from a subject. Some embodiments concern polypeptides for use in generating standard curves for an immunoassay. Other embodiments provide methods and kits for detecting precursor THP in a biological sample. Further provided are methods for predicting the risk of a subject (e.g., a human) for developing acute kidney injury or chronic kidney disease.

### CROSS-REFERENCE TO RELATED APPLICATIONS

This application is a 371 U.S. National Phase of International PCT Application No. PCT/US2022/012457, filed Jan. 14, 2022 which claims priority to U.S. Provisional Application No. 63/137,258, filed on Jan. 14, 2021, and U.S. Provisional Application No. 63/148,897, filed on Feb. 12, 2021, the entire disclosures of which are hereby expressly incorporated by reference in their entirety.

### STATEMENT OF GOVERNMENT SUPPORT

This invention was made with government support under DK111651 awarded by National Institutes of Health and BX003935 merit award by the Veterans Administration. The Government has certain rights in the invention.

### REFERENCE TO SEQUENCE LISTING

The instant application contains a Sequence Listing which has been submitted electronically and is hereby incorporated by reference in its entirety. Said.txt copy, created on Feb. 18, 2022, is named “IU-2021-043-03-WO_SL.txt” and is 13 KB in size. The Sequence Listing is also provided in the following table:

TABLE of
Sequences

| | | SEQ |
|---|---|---|
| | | ID |
| Description | Sequence | NO |
| Precursor | MGQPSLTWML MVVVASWFIT TAATDTSEAR | 1 |
| THP | WCSECHSNAT CTEDEAVTTC TCQEGFTGDG | |
| | LTCVDLDECA IPGAHNCSAN SSCVNTPGSF | |
| | SCVCPEGFRL SPGLGCTDVD ECAEPGLSHC | |
| | HALATCVNVV GSYLCVCPAG YRGDGWHCEC | |
| | SPGSCGPGLD CVPEGDALVC ADPCQAHRTL | |
| | DEYWRSTEYG EGYACDTDLR GWYRFVGQGG | |
| | ARMAETCVPV LRCNTAAPMW LNGTHPSSDE | |
| | GIVSRKACAH WSGHCCLWDA SVQVKACAGG | |
| | YYVYNLTAPP ECHLAYCTDP SSVEGTCEEC | |
| | SIDEDCKSNN GRWHCQCKQD FNITDISLLE | |
| | HRLECGANDM KVSLGKCQLK SLGFDKVFMY | |
| | LSDSRCSGFN DRDNRDWVSV VTPARDGPCG | |
| | TVLTRNETHA TYSNTLYLAD EIIIRDLNIK | |
| | INFACSYPLD MKVSLKTALQ PMVSALNIRV | |
| | GGTGMFTVRM ALFQTPSYTQ PYQGSSVTLS | |
| | TEAFLYVGTM LDGGDLSRFA LLMTNCYATP | |
| | SSNATDPLKY FIIQDRCPHT RDSTIQVVEN | |
| | GESSQGRFSV QMFRFAGNYD LVYLDCEVYL | |
| | CDTMNEKCKP TCSGTRFRSG SVIDQSRVLN | |
| | LGPITRKGVQ ATVSRAFSSL GLLKVWLPLL | |
| | LSATLTLTFQ | |
| Mature THP | MGQPSLTWML MVVVASWFIT TAATDTSEAR | 2 |
| | WCSECHSNAT CTEDEAVTTC TCQEGFTGDG | |
| | LTCVDLDECA IPGAHNCSAN SSCVNTPGSF | |
| | SCVCPEGFRL SPGLGCTDVD ECAEPGLSHC | |
| | HALATCVNVV GSYLCVCPAG YRGDGWHCEC | |
| | SPGSCGPGLD CVPEGDALVC ADPCQAHRTL | |
| | DEYWRSTEYG EGYACDTDLR GWYRFVGQGG | |
| | ARMAETCVPV LRCNTAAPMW LNGTHPSSDE | |
| | GIVSRKACAH WSGHCCLWDA SVQVKACAGG | |
| | YYVYNLTAPP ECHLAYCTDP SSVEGTCEEC | |
| | SIDEDCKSNN GRWHCQCKQD FNITDISLLE | |
| | HRLECGANDM KVSLGKCQLK SLGFDKVFMY | |
| | LSDSRCSGFN DRDNRDWVSV VTPARDGPCG | |
| | TVLTRNETHA TYSNTLYLAD EIIIRDLNIK | |
| | INFACSYPLD MKVSLKTALQ PMVSALNIRV | |
| | GGTGMFTVRM ALFQTPSYTQ PYQGSSVTLS | |
| | TEAFLYVGTM LDGGDLSRFA LLMTNCYATP | |
| | SSNATDPLKY FIIQDRCPHT RDSTIQVVEN | |
| | GESSQGRFSV QMFRFAGNYD LVYLDCEVYL | |
| | CDTMNEKCKP TCSGTRF | |
| Precursor THP | SVIDQSRVLNLGPITRKGVQAT | 3 |
| epitope | | |
| EHP domain | VLNLGPITRK | 4 |
| Mature THP | DRDNRDWVSVVTPAR | 5 |
| epitope | | |
| Calibration | DRDNRDWVSVVTPARSVIDQSRVLNLGPITR | 6 |
| polypeptide | KGVQAT | |
| (Gly)₈ | GGGGGGGG | 7 |
| Linker | | |
| GS Linker | (Gly-Gly-Gly-Gly-Ser)n | 8 |
| Linker | Gly-Ser-Ala-Gly-Ser-Ala- | 9 |
| | Ala-Gly-Ser-Gly-Glu-Phe | |
| Anti-mature | DRDNRDWXSXXTPAR | 10 |
| THP antibody | | |
| recognition | | |
| sequence | | |

### FIELD AND BACKGROUND

Tamm-Horsfall protein (THP) is a glycoprotein uniquely produced by the epithelial cells lining the thick ascending limb (TAL) of Henle's loop and early distal convoluted tubule (DCT). Its exclusive renal expression, great urinary abundance and phylogenetic conservation point to the important physiological roles that are of interest to researchers and clinicians. THP is predominantly sorted to the apex of TAL cells and secreted into the urine as one of the most abundant proteins. A lesser, but significant amount of THP is released basolaterally, towards the interstitium and circulation. Circulating levels of THP are substantially lower than urinary levels (ng/ml versus ug/ml, respectively). In the last two decades, important advancements have been made to delineate the biosynthesis, intracellular maturation along the secretory pathway, excretion and polymerization of mature urinary THP.

Recent discoveries have underscored the importance of THP (encoded by the UMOD gene) as a regulatory protein in health and in various pathological conditions, such as medullary cystic kidney disease, glomerulocystic kidney disease, urinary tract infections, nephrolithiasis, and acute kidney injury

### SUMMARY

In a first example (“Example 1), provided herein is a method for predicting a subject's risk for acute kidney injury or risk for chronic kidney disease, comprising: a) detecting a level of precursor Tamm-Horsfall protein (THP) in a biological sample obtained from the subject; and b) identifying the subject as having an increased risk of acute kidney injury or risk of chronic kidney disease when the detected level of precursor THP in the biological sample obtained from the subject is lower than mean levels of precursor THP in biological samples from a control population.

In another example (“Example 2”), further to Example 1, the step of detecting the level of precursor THP comprises: a) contacting the biological sample obtained from the subject with an anti-precursor THP antibody; and b) detecting binding of the anti-precursor THP antibody to precursor THP present in the biological sample obtained from the subject.

In another example (“Example 3”), further to Example 1, the step of detecting the level of precursor THP comprises: a) contacting the biological sample with a capture antibody, wherein the capture antibody is an anti-mature THP antibody or an anti-precursor THP antibody; b) incubating the biological sample and the capture antibody to allow for formation of precursor THP-capture antibody complexes; c) adding a detection antibody following incubation, wherein the detection antibody is an anti-mature THP antibody wherein the capture antibody is an anti-precursor THP antibody, or is an anti-precursor THP antibody wherein the capture antibody is an anti-mature THP antibody; and d) detecting binding of the detection antibody to formed precursor THP-capture antibody complexes.

In another example (“Example 4”), further to Example 2 or Example 3, the anti-precursor THP antibody binds to an external hydrophobic patch (EHP) domain of precursor THP.

In another example (“Example 5”), further to any one of Examples 2-4, anti-precursor THP antibody binds to an amino acid sequence of SEQ ID NO: 3 (SVIDQSRVLNLGPITRKGVQAT).

In another example (“Example 6”), further to any one of Examples 2-5, the anti-precursor THP antibody does not bind mature THP.

In another example (“Example 7”), further to any one of Examples 1-7, detecting a level of precursor THP in a biological sample obtained from the subject comprises use of a direct enzyme-linked immunosorbent assay (ELISA), an indirect ELISA, a competitive ELISA, a sandwich ELISA, or an amplified luminescent proximity homogenous assay (AlphaLISA®).

In another example (“Example 8”), further to any one of Examples 1-7, the biological sample is a blood serum or plasma sample, or a urine sample

In another example (“Example 9”), provided herein is a polypeptide comprising a first domain comprising an amino acid sequence having at least 95% sequence identity to SVIDQSRVLNLGPITRKGVQAT (SEQ ID NO: 3), and a second domain comprising an epitope of mature Tamm-Horsfall protein (THP), wherein the first domain and the second domain are directly joined together, or are separated by 1 to 100 amino acids.

In another example (“Example 10”), further to Example 10, the second domain comprises an amino acid sequence having at least 95% sequence identity to DRDNRDWVSVVTPAR (SEQ ID NO: 5).

In another example (“Example 11”), provided herein is a polypeptide comprising an amino acid sequence having at least 95% sequence identity to DRDNRDWVSVVTPARSVIDQSRVLNLGPITRKGVQAT (SEQ ID NO: 6).

In another example (“Example 12”), provided herein is a polypeptide having at least 95% sequence identity to DRDNRDWVSVVTPARSVIDQSRVLNLGPITRKGVQAT (SEQ ID NO: 6).

In another example (“Example 13”), further to any one of Examples 9-11, the polypeptide includes a flexible linker between the first domain and the second domain.

In another example (“Example 14”), further to any one of Examples 9-13, the at least 95% sequence identity is at least 98% sequence identity.

In another example (“Example 15”), further to any one of Examples 9-14, the polypeptide is capable of binding to a mature Tamm-Horsfall protein (THP) antibody and an anti-precursor THP antibody.

In another example (“Example 16”), provided herein is a polynucleotide encoding the polypeptide of any one of Examples 9-15.

In another example (“Example 17”), provided herein is an expression vector comprising the polynucleotide of Example 16.

In another example, (“Example 18), provided herein is a cell comprising the expression vector of Example 17.

In another example (“Example 19”), provided herein are methods for generating a standard curve for an immunoassay for detecting precursor Tamm-Horsfall protein (THP) in a biological sample, the method comprising: a) adding a known concentration of the polypeptide of any one of claims 9-15 to a container comprising a capture antibody, wherein the capture antibody is an anti-THP antibody or an anti-precursor THP external hydrophobic patch (EHP) antibody; b) incubating the polypeptide and the capture antibody to form polypeptide-capture antibody complexes; c) adding a detection antibody to the polypeptide-capture antibody complex, wherein the detection antibody is an anti-mature THP antibody wherein the capture antibody is an anti-precursor THP antibody, or is an anti-precursor THP antibody wherein the capture antibody is an anti-mature THP antibody; and d) detecting binding of the detection antibody to the polypeptide-capture antibody complex.

In another example (“Example 20”), further to Example 20, the method includes the steps of: e) repeating steps a) through d) using varying known concentrations of the polypeptide; and f) generating a standard curve based upon the binding detected between the detection antibody and the polypeptide-capture antibody complex at the varying known concentrations of the polypeptide.

In another example (“Example 21”), further to Example 19 or Example 20, the anti-precursor THP antibody binds to an amino acid sequence of SEQ ID NO: 3

(SVIDQSRVLNLGPITRKGVQAT).

In another example (“Example 22”), further to any one of Examples 19-21, the capture antibody is affixed within a well of a microplate.

In another example (“Example 23”), further to any one of Examples 19-22, the immunoassay is an enzyme-linked immunosorbent assay (ELISA) or an amplified luminescent proximity homogenous assay (AlphaLISA®).

In another example (“Example 24”), provided herein is a method for quantifying precursor Tamm-Horsfall protein (THP) in a biological sample, the method comprising: a) contacting the biological sample with a capture antibody, wherein the capture antibody is an anti-THP antibody or an anti-precursor THP antibody; b) incubating the biological sample and the capture antibody to allow for formation of precursor THP-capture antibody complexes; c) adding a detection antibody following incubation, wherein the detection antibody is an anti-THP antibody wherein the capture antibody is an anti-precursor THP antibody, or is an anti-precursor THP antibody wherein the capture antibody is an anti-THP antibody; and d) detecting binding of the detection antibody to any formed precursor THP-capture antibody complexes.

In another example (“Example 25”), further to Example 24 the capture antibody is an anti-THP antibody and the detection antibody is an anti-precursor THP antibody.

In another example (“Example 26”), further to Example 24 or Example 25, the method includes comparing an amount of binding detected between the detection antibody and any formed precursor THP-capture antibody complexes to a standard curve generated in accordance with any one of Examples 19-23

In another example (“Example 27”), further to any one of Examples 24-26, the biological sample is a blood serum or plasma sample, or a urine sample.

In another example (“Example 28”), further to any one of Examples 24-27, the method includes identifying a subject from which the biological sample was obtained as having an increased risk of developing acute kidney injury or chronic kidney disease when precursor THP is detected in the biological sample at a lower level than mean levels of precursor THP detected in biological samples from a control population.

In another example (“Example 28”), provided herein is a kit for performing an ELISA or an AlphaLISA® to detect precursor THP in a sample, comprising a polypeptide of any one of Examples 9-15.

In another example (“Example 29”), further to Example 28, the kit includes a capture antibody and a detection antibody.

In another example (“Example 30”), further to Example 28, the capture antibody is an anti-mature THP antibody or an anti-precursor THP antibody; and wherein the detection antibody is an anti-mature THP antibody when the capture antibody is an anti-precursor THP antibody, or is an anti-precursor THP antibody wherein the capture antibody is an anti-mature THP antibody.

In another example (“Example 31”), further to any one of Examples 29-30, the capture antibody is an anti-mature THP antibody and the detection antibody is an anti-precursor THP antibody.

### BRIEF DESCRIPTION OF THE DRAWINGS

The following drawings form part of the present specification and are included to further demonstrate certain embodiments. Some embodiments may be better understood by reference to one or more of these drawings alone or in combination with the detailed description of specific embodiments presented.

FIG. 1A depicts a schematic diagram of THP structure and its functional domains. The top panel shows the initial 640 amino acid THP precursor and the bottom panel its processed mature urinary form. Abbreviations: SP—signal peptide; I, II, III and IV—EGF-like domains; D8C—cysteine rich domain; ZP—zona pellucida domain; GPI—glycosylphosphatidylinositol anchor at 614; CCS—consensus cleavage site at 585; IHP—internal hydrophobic patch (430-436 and 456-462 of SEQ ID NO:1); EHP—external hydrophobic patch (598-607 of SEQ ID NO: 1).

FIG. 1B depicts the amino acid sequence of full-length precursor THP (SEQ ID NO: 1) with color coded domain annotation: red—signal peptide; green—EFG-like domains; blue—D8C disulfide bond domain; purple—ZP-N and ZP-C domains; lavender magenta—EHP; light green—IHP; yellow—CCS; cyan—GPI anchor attachment site; magenta—sites of N-linked glycosylation.

FIG. 2A depicts a schematic representation of precursor THP with annotation of its functional domains, focusing on the C-terminus (enlarged; amino acids 581-640 of SEQ ID NO: 1). An antibody against the indicated sequence encompassing the external hydrophobic patch (EHP) was developed.

FIG. 2B depicts photographs of denaturing and native Western blots. The left panel depicts a denaturing Western blot where THP with retained EHP (precursor THP) is detected by anti-precursor THP antibody in the urine (U) and mature urinary THP is detected with an antibody that was raised against mature THP (anti-THP). No EHP signal is detected in mature THP that was purified based on its aggregative properties (pTHP). Native separate immunoblots revealed that THP with retained EHP is a dimer in its native form (arrowhead, right panel), whereas mature urinary THP without EHP polymerizes into high molecular weight multimers (arrows, right panel).

FIG. 2C presents representative immunofluorescence confocal microscopy data of a human kidney sectioned and stained to detect THP and EHP. Representative images (high and low magnifications, left and right, respectively) are shown (scale bars=100 μm). Precursor THP with EHP colocalizes with mature THP in the thick ascending limbs (TAL) tubules, where it is maximally expressed at the apical domain (white arrows). However, a significant, albeit less intense EHP signal, can also be detected in other segments such as proximal tubules (arrowhead). Quantitation of EHP signal per tubule type from 3 separate fields is depicted in the graph. Asterisks denotes P<0.001

FIG. 3A depicts the design of a sandwich ELISA for measurement of precursor THP. Epitope mapping was performed for a commercially available monoclonal antibody (Anti-THP (m)) and a synthetic hybrid peptide combining the recognizable sequence in mature THP along with EHP sequence was generated for calibration. A representative standard curve using the hybrid peptide is shown in the panel on the right. FIG. 3A depicts the full-length sequence for precursor THP (SEQ ID NO: 1), SEQ ID NO: 10, an anti-mature THP antibody recognition sequence (DRDNRDWXSXXTPAR; amino acids 371-385 of SEQ ID NO:1), and an anti-precursor THP antibody recognition sequence (SVIDQSRVLNLGPITRKGVQAT; amino acids 591-612 of SEQ ID NO: 1, and SEQ ID NO: 3)

FIG. 3B presents data indicating levels of precursor THP (THP+EHP) and total THP in the urine of a cohort of 20 patients with cirrhosis measured on hospital admission. Total and precursor THP did not have a direct significant correlation (left panel). After adjustment to total urinary proteins, only precursor THP, but not total THP, was lower in patients who subsequently developed AKI (middle and right panels).

FIG. 3C presents data indicating successful measurement of precursor THP in the plasma, albeit at low levels. Levels did not associate with kidney injury.

FIG. 4 depicts an immunoblot of immunoprecipitated serum and urine THP. Immunoprecipitation (IP) of serum THP was performed, followed by Western blot and compared to urine THP. Serum THP is of slightly higher molecular weight (arrow) compared to urine THP. A control IP reaction (i.e., with non-immune IgG) is shown in lane to the left of the immunoprecipitated serum THP.

### DETAILED DESCRIPTION

Embodiments herein provide compositions, methods, uses for detecting precursor Tamm-Horsfall protein (THP) in a biological sample from a subject. Some embodiments concern polypeptides for use in generating standard curves for an immunoassay. Other embodiments provide methods and kits for detecting precursor THP in a biological sample. Further provided are methods for predicting the risk of a subject (e.g., a human) for developing acute kidney injury or chronic kidney disease.

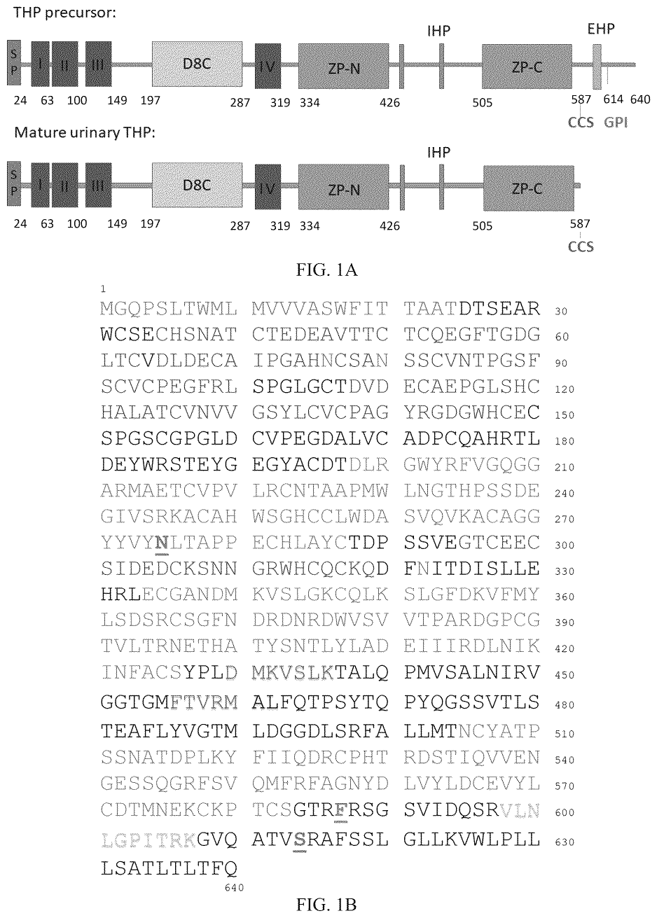

Tamm-Horsfall protein is synthesized as a 640 amino acid precursor protein (“precursor THP”) (FIG. 1A, top panel; SEQ ID NO: 1). The precursor protein moves through the secretory pathway where it is glycosylphosphatidylinositol anchored, glycosylated, and sorted to the apical plasma membrane of epithelial cells. Referring to FIG. 1A (top panel), the precursor protein includes a leader polypeptide directing its entry into the secretory pathway (SP), four epidermal growth factor (EGF)-like domains (I, II, III, and IV), a central domain of unknown function including eight conserved cysteines (DC8), and a bipartite zona pellucida (ZP) domain essential for THP assembly into extracellular urinary polymers of supramolecular structure. Extensive hydrophobic interface mediates the ZP-N domain homodimerization, while a structured interdomain linker between ZP-N and ZP-C directs that self-association. During intracellular trafficking, precursor THP is kept in a polymerization-incompetent state by the hydrophobic interaction of two motifs, the so-called internal hydrophobic patch (IHP, residues 430-436 and 456-462 of SEQ ID NO: 1) and external hydrophobic patch (EHP, residues 598-607 of SEQ ID NO: 1). IHP is located in the ZP linker region, while the EHP resides between the consensus proteolytic cleavage site (F587 of SEQ ID NO: 1) and GPI anchoring site (S614 of SEQ ID NO: 1) at the apical membrane on the urinary lumen side. Proteolysis by type II transmembrane serine protease hepsin at conserved F587, removes the EHP motif, thus permitting correct orientation of ZP-N domains for polymerization to occur in the urine.

As described herein, precursor THP can be detected in serum and urine samples. Detection is facilitated by a novel selective anti-precursor THP antibody, as well as by the development of a novel assay calibration polypeptide. Precursor THP levels are demonstrated herein to be linked to the risk for acute kidney injury and chronic kidney disease.

### Calibration Polypeptides

Polypeptides are provided that can be used for assay calibration. The assay to be calibrated can be an immunoassay such as an enzyme-linked immunosorbent assay (ELISA) or an amplified luminescent proximity homogenous assay (AlphaLISA®). The polypeptides allow for the production of a standard curve for the immunoassay. The polypeptide includes two domains: a first domain including an amino acid sequence that includes the precursor THP external hydrophobic patch (EHP) domain; and a second domain including an epitope of the mature THP. The first and second domains can be either directly joined together, or separated by 1 to 100 amino acids. The first and second domains can be separated by 100 amino acids, fewer than 100 amino acids, fewer than 90 amino acids, fewer than 80 amino acids, fewer than 70 amino acids, fewer than 60 amino acids, fewer than 50 amino acids, fewer than 40 amino acids, fewer than 30 amino acids, fewer than 20 amino acids, fewer than 10 amino acids, 9 amino acids, 8 amino acids, 7 amino acids, 6 amino acids, 5 amino acids, 4 amino acids, 3 amino acids, 2 amino acids, or 1 amino acid.

The first domain includes the amino acid sequence of the EHP domain of precursor THP (VLNLGPITRK, SEQ ID NO: 4; amino acids 598-607 of SEQ ID NO: 1). The first domain can include an amino acid sequence having at least 90% sequence identity to SEQ ID NO: 3 (SVIDQSRVLNLGPITRKGVQAT; amino acids 591-612 of SEQ ID NO: 1).

The second domain includes an epitope of the mature THP. The mature THP can be a mature human THP. Several anti-mature THP monoclonal antibodies are commercially available. Through epitope mapping, it is possible to identify the epitope of mature THP to which a particular anti-mature THP antibody binds. An identified mature THP epitope can be incorporated into the described calibration polypeptide as part of the second domain. The second domain can include an amino acid sequence having at least 90% sequence identity to SEQ ID NO: 5 (DRDNRDWVSVVTPAR).

The first and second domains can occur in any order. That is, either the first domain or the second domain may appear at or near the N-terminus of the polypeptide. In some embodiments the order of the two domains, from N-terminus to C-terminus, is the first domain followed by the second domain. In other embodiments, the order of the two domains, from N-terminus to C-terminus, is the second domain followed by the first domain.

The calibration polypeptide can include an amino acid sequence having at least 90% sequence identity to SEQ ID NO: 6 (DRDNRDWVSVVTPARSVIDQSRVLNLGPITRKG VQAT). SEQ ID NO: 6 includes both first (SEQ ID NO: 3, SVIDQSRVLNLGPITRKGVQAT) and second (SEQ ID NO: 5, DRDNRDWVSVVTPAR) domains. In some embodiments, the calibration polypeptide consists of an amino acid sequence having at least 90% sequence identity to SEQ ID NO: 6.

In some embodiments, the calibration polypeptide can include an amino acid linker between the first and second domains. Suitable linkers are known in the art and are generally rich in small or polar amino acids such as glycine and serine to provide good flexibility and solubility. Suitable linkers link the first and second domains together without introducing any tertiary protein structure, or otherwise affect each domain's ability to bind an antibody. Examples of suitable linkers include glycine repeat linkers ((Gly) n; e.g., Gly-Gly, and (Gly)₈ (SEQ ID NO: 7)) and “GS” linkers primarily made up of stretches of glycine and serine (e.g., (Gly-Gly-Gly-Gly-Ser)ₙ (SEQ ID NO: 8)), although others are also known (e.g., Gly-Ser-Ala-Gly-Ser-Ala-Ala-Gly-Ser-Gly-Glu-Phe (SEQ ID NO: 9)). Linkers between the first and second domains should be kept to 100 amino acids or less. The linker between the first and second domains can be 100 amino acids in length, fewer than 100 amino acids, fewer than 90 amino acids, fewer than 80 amino acids, fewer than 70 amino acids, fewer than 60 amino acids, fewer than 50 amino acids, fewer than 40 amino acids, fewer than 30 amino acids, fewer than 20 amino acids, fewer than 10 amino acids, 9 amino acids, 8 amino acids, 7 amino acids, 6 amino acids, 5 amino acids, 4 amino acids, 3 amino acids, 2 amino acids, or 1 amino acid in length. As provided above, a linker is not required to link the first and second domains, but can be beneficial in certain situations to promote antibody binding.

The polypeptide disclosed herein is capable of binding to a specific anti-precursor THP antibody and an anti-mature THP antibody. In some embodiments, the specific anti-precursor THP antibody binds at least the EHP domain of precursor THP (VLNLGPITRK, SEQ ID NO: 4; amino acids 598-607 of SEQ ID NO: 1). The precursor THP epitope can be, for example, SEQ ID NO: 3 (SVIDQSRVLNLGPITRKGVQAT; amino acids 591-612 of SEQ ID NO: 1). Several anti-mature THP antibodies, both monoclonal and polyclonal, are commercially available (e.g., Santa Cruz Biotechnology sc-271022, a mouse anti-mature THP polyclonal antibody). The second domain includes a mature THP epitope, thus providing a binding site for an anti-mature THP antibody. The second domain can include the amino acid sequence DRDNRDWVSVVTPAR (SEQ ID NO: 5), which was determined by epitope mapping to be an epitope for sc-271022antibody. Wherein a different anti-mature THP antibody is to be used, an appropriate epitope can be included in the second polypeptide domain.

The at least 90% sequence identity can be at least 95% sequence identity, at least 98% sequence identity, at least 99% sequence identity, or 100% sequence identity. Sequence identity can be determined in several ways that are within the skill of a person skilled in the art. Examples of tools useful for calculating sequence identity include publicly available computer software such as BLAST, BLAST-2, ALIGN, and Megalign software. Details regarding the algorithms used by these software programs are known in the art. Those skilled in the art can determine appropriate parameters for measuring alignment, including any algorithms needed to achieve maximal alignment over the full length of the sequences being compared.

Methods for producing the polypeptides disclosed here are known in the art, and are within the skill of the person skilled in the art of the present disclosure. The polypeptides can be synthesized chemically, or by recombinant DNA methods. The polypeptides can be chemically synthesized using techniques such as liquid-phase or solid-phase peptide synthesis. In other embodiments, DNA having nucleic acid sequences encoding the polypeptides disclosed herein can be synthesized chemically or by recombinant DNA methodologies, and expressed via a recombinant expression system to produce the polypeptides. The DNA sequence encoding the polypeptide can be cloned according to standard recombinant DNA methodologies. The resulting DNA molecules can be ligated to other appropriate nucleic acid sequences, such as expression control sequences (e.g., promoters) and plasmids, to produce a gene expression construct such as an expression vector that encodes the desired polypeptide.

Expression vectors encoding the desired polypeptide can be introduced into host cells through conventional transfection or transformation techniques. Host can be, for example, one of E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the gene that encodes the desired polypeptide. Following expression, the polypeptide can be collected and purified or isolated using techniques such known in the art.

Polynucleotides encoding a polypeptide described herein are contemplated by the present disclosure, as are expression vectors comprising such polynucleotides, and cells comprising such expression vectors.

### Detection of Precursor-THP

The concentration of precursor THP can be quantitated in a bodily fluid sample by measuring binding of precursor THP by anti-precursor THP antibodies. Such binding can be measured by, for example, an immunoassay or a flow-through assay. The methods and compositions described herein can be used to detect the concentration of precursor THP in a sample, such as a blood serum, blood plasma, or urine sample, for the evaluation of risk for acute kidney injury or chronic kidney disease in a subject.

Immunoassays of use in quantifying the concentration of precursor THP in a bodily fluid sample include the enzyme-linked immunosorbent assays (ELISAs), such as the direct ELISA, indirect ELISA, competitive ELISA, and sandwich ELISA.

In the direct ELISA, sample is applied to a protein-binding solid surface such as a microtiter plate, resulting in the analyte (i.e., premature THP) being immobilize onto the surface. A reporter-conjugated detection (primary) antibody specific for the analyte is then contacted with the immobilized proteins from the sample. Signal from the detection antibody is measured and analyzed to provide a quantitative result when used with a standard curve of known concentration.

In the indirect ELISA, sample is applied to a protein-binding solid surface such as a microtiter plate, resulting in the analyte (i.e., premature THP) being immobilized onto the surface, as in the direct ELISA. A detection antibody, or primary antibody, specific for the analyte is then contacted with the immobilized proteins from the sample. Non-binding proteins can be blocked, and a reporter-conjugated reporter (secondary) antibody is used to detect the bound analyte-specific antibody. Signal from the directly conjugated reporter molecule is measured and analyzed to provide a quantitative result when used with a standard curve of known concentration.

In the competitive ELISA, the sample analyte (i.e., premature THP) competes with a reference analyte for binding to a specific amount of labeled detection antibody. The reference analyte is pre-coated on a solid surface such as a microtiter plate, and sample is pre-incubated with reporter-conjugated detection antibody. The sample/antibody combination is then contacted with the reference analyte. Depending on the amount of sample analyte in the sample, more of less free antibodies will be available to bind the reference antigen. The more analyte in the sample, the less reference antigen will be detected and the weaker the detected signal. Alternatively, the reference analyte may be labeled.

In the sandwich ELISA, a capture antibody is immobilized on a solid surface, such as a microtiter plate. The biological sample (i.e., the fluid sample) is then contacted with the capture antibody. The biological sample is incubated with the capture antibody to allow for formation of analyte (i.e., precursor THP)-capture antibody complexes. A detection antibody is then added following the incubation, wherein the detection antibody binds a second epitope on the analyte. Binding of the detection antibody to the analyte is then detected either directly using a reporter-conjugated detection antibody, or indirectly through the use of a reporter antibody having a reporter molecule conjugated thereto, where binding of the reporter antibody to the detection antibody is detected. Signal from the detection antibody or detection antibody/reporter antibody complex is measured and analyzed to provide a quantitative result when used with a standard curve of known concentration. The detected and measured binding is compared to the standard curve, allowing for quantification of analyte (i.e., precursor THP) in the biological sample.

In the sandwich ELISA, the capture and detection antibodies can be contacted with the biological sample simultaneously or sequentially.

The capture antibody can be an anti-mature THP antibody. Using an anti-mature THP antibody will result in all THP present in a sample (i.e., mature THP and precursor THP) being bound by the capture antibody. This is because precursor THP includes the entirety of the mature THP; it is the C terminus of the precursor antibody that is proteolytically cleaved to produce the mature THP. When an anti-mature THP antibody is used as the capture antibody, an anti-precursor antibody is used as the detection antibody. This provides for selective detection of precursor THP in the sample. In certain embodiments, using an anti-mature THP monoclonal antibody as the capture antibody results in higher specificity in the sandwich ELISA configuration.

Alternatively, the capture antibody can be an anti-precursor THP antibody. Using an anti-precursor antibody will result in only precursor THP present in the sample being bound by the capture antibody. Since only precursor THP is captured, an anti-mature THP antibody can then be used as the detection antibody to provide for detection of precursor THP in the sample.

As provided above, the detection antibody can either be directly conjugated with a detection molecule, or can itself be detected utilizing a reporter antibody.

The various ELISAs each have their advantages and disadvantages, which will be recognized and acknowledged by those skilled in the art. For example, the direct ELISA is faster than other ELISA techniques, and using less reagents. However, because the method of immobilizing the analyte is not specific, the method is prone to high background noise as all proteins in the sample, including the analyte, will bind to the plate. The indirect ELISA has a higher sensitivity than the direct ELISA, as more than one labeled reporter antibody can bind the detection antibody. However, use of the reporter antibody can increase background noise to cross-reactivity with non-analyte immobilized proteins. The design of the competitive ELISA requires no sample processing, allowing for the use of crude or impure samples. The competitive ELISA is generally used when only one antibody is available for the analyte of interest, or when the target analyte is small and does not provide for binding by two different antibodies. The sandwich ELISA is both highly sensitive and selective, and offers flexibility as both direct and indirect methods can be used. The sandwich ELISA is particularly suitable for the analysis of complex samples, since the analyte of interest does not need to be purified. This is of particular importance with precursor THP, which is very difficult to isolate and purify. In particular embodiments, a sandwich ELISA is used.

Detection strategies for ELISAs are known in the art, and can be based on chromogenic (colorimetric), fluorescence, or chemiluminescence methodologies. The detection molecule can be alkaline phosphatase (AP), horseradish peroxidase (HRP), or a fluorescent tag that can be directly observed (e.g., Cy3 and Cy5), fluorescein isothiocyanate, rhodamine, phycoerytherin, phycocyanin, allophycocyanin, o-phthalaldehyde and fluorescamine).

Chromogenic substrates are available for both alkaline phosphatase (para-nitrophenyl phosphate (PNPP)) and horseradish peroxidase (3,3′,5,5′-tetramethylbenzidine (TMB), o-phenylenediamine dihydrochloride (OPD), and 2,2′-azino-bis(3-ethylbenzothiazoline-6-sulphonic acid (ABTS)). The chromogenic substrate is added following the reporter antibody, and form a soluble, colored product that accumulates over time relative to the amount of enzyme present. When the desired color intensity is reached, the product absorbance is either measured directly or a stop solution is added to provide a fixed end-point for the assay.

For chemiluminescent detection, a luminol-based substrate can be used in combination with HRP and a peroxide buffer. Chemiluminescent substrates for AP are also available and can be used. While chemiluminescent detection may be more sensitive than colorimetric detection, signal intensity is known to vary more than with other substrates.

Detection may also be accomplished using a radioactively labeled antibody. It is then possible to detect the antibody through the use of radioimmune assays. The radioactive isotope can be detected by such means as the use of a gamma counter or a scintillation counter or by autoradiography. Isotopes which are particularly useful for the purpose of the present invention are 3H, 131I, 35S, 14C, and 125I.

Signal amplification can be achieved using biotinylated detection or reporter antibodies according to methods known in the art.

A variation of the ELISA, known as an amplified luminescent proximity homogenous assay (AlphaLISA®; PerkinElmer, Waltham, Mass.), can also be used to detect levels of precursor THP in a biological sample. In the AlphaLISA® assay, streptavidin-coated donor beads containing a photosensitizer are added to a mixture of analyte (i.e., precursor THP), a first biotinylated antibody that recognizes the analyte, and acceptor beads conjugates to a second antibody that also recognizes the analyte. The AlphaLISA® assay uses luminescent oxygen-channeling chemistry in which laser irradiation of donor beads causes chemiluminescent emission from the acceptor bead. Thus, when the analyte is present, the proximity of the donor and acceptor beads triggers a reaction that results in chemiluminescent emission from the acceptor bead. The signal generated is proportional to the amount of analyte in the sample.

Although neither antibody acts as a “capture” antibody, as described herein, the antibody that is biotinylated for use in the AlphaLISA® assay is referred to herein as the “capture antibody”, while the acceptor bead-conjugated antibody is referred to herein as the “detection antibody”. Either the anti-mature THP or anti-precursor THP antibody can be biotinylated to generate the capture antibody. Likewise, either antibody can be conjugated to the acceptor beads to generate the detection antibody. As provided above, when the capture antibody is anti-mature THP, the detection antibody is anti-precursor THP. When the capture antibody is anti-precursor THP, the detection antibody is anti-mature THP.

### Antibodies

In some embodiments, the anti-mature THP antibodies are monoclonal antibodies and the anti-precursor THP antibodies are polyclonal. Monoclonal and polyclonal antibodies can be prepared using a variety of techniques known in the art. As provided above, several monoclonal anti-mature THP antibodies are commercially available.

Polyclonal anti-precursor THP antibodies can be generated by immunizing an animal with a fragment of THP specific to the precursor form of the protein, such as one that includes the EHP domain. For example, polyclonal anti-precursor THP antibodies can be generated by injection a lab animal such a rabbit or a goat with a polypeptide including the amino acid sequence of SEQ ID NO: 3 (SVIDQSRVLNLGPITRKGVQAT), or an amino acid sequence having at least 95% sequence identity to SEQ ID NO: 3. The amino acid sequence of SEQ ID NO: 3 or at least 95% identical thereto may, for example, be conjugated to a larger carrier protein such as keyhole limpet hemocyanin (KLH), bovine serum albumin (BSA), or ovalbumin (OVA). Because the described polypeptide itself is likely too small to elicit a sufficient immune response, a carrier protein including several epitopes can be used to stimulate T-helper cells, which subsequently facilitates the induction of the B-cell response and thus antibody production.

### Standard Curves

Methods for generating a standard curve for an ELISA or an AlphaLISA are provided. Such methods include adding a known concentration of a polypeptide described herein to a capture antibody, incubating the polypeptide and the capture antibody to form polypeptide-capture antibody complexes, adding a detection antibody, and detecting binding of the detection antibody to the polypeptide-capture antibody complex. These steps can be repeated using varying known concentrations of the polypeptide and generating a standard curve based upon the binding of the polypeptide at the varying concentrations. The method can also include comparing the amount of the binding of the detection antibody to the precursor THP-capture antibody complexes to a standard curve generated as described herein. This allows for quantification of precursor THP in a biological sample.

In some embodiments, the standard curve is generated by adding the polypeptide to a container including an anti-mature THP antibody or to a surface to which the anti-mature THP antibody is affixed, allowing the polypeptide to bind the anti-mature THP antibody to form a polypeptide-anti-mature THP antibody complex, adding an anti-precursor THP antibody to the polypeptide-anti-mature THP antibody complex, and detecting binding of the anti-precursor THP antibody to the polypeptide-anti-mature THP antibody complex. Alternatively, the polypeptide can be added to a container including an anti-precursor THP antibody or to a surface to which the anti-precursor THP antibody is affixed, allowing the polypeptide to bind the anti-precursor THP antibody to form a polypeptide-anti-precursor THP antibody complex, adding an anti-mature THP antibody to the polypeptide-anti-precursor THP antibody complex, and detecting binding of the anti-mature THP antibody to the polypeptide-anti-precursor THP antibody complex.

### Determining Risk of Acute Kidney Injury or Chronic Kidney Disease

As described herein, low levels of precursor THP either in the blood serum, blood plasma, or urine are correlated with a subject having an increased risk of acute kidney injury or chronic kidney disease. Provided are methods for predicting a subject's risk for acute kidney injury or risk for chronic kidney disease.

Methods for predicting a subject's risk for acute kidney injury or risk for chronic kidney disease include detecting a level of precursor THP in a biological sample obtained from the subject and identifying the subject as having an increased risk of acute kidney injury or risk of chronic kidney disease when the detected level of precursor THP in the biological sample obtained from the subject is lower than levels of precursor THP in biological samples from a control population. In some embodiments, the subject is identified as having an increased risk for acute kidney injury or risk of chronic kidney disease when the detected level of precursor THP in the biological sample obtained from the subject is lower than mean levels of precursor THP in biological samples from a control population.

The detecting step can include contacting the biological sample from the subject with an anti-precursor THP antibody and detecting binding of the anti-precursor THP antibody to precursor THP present in the biological sample obtained from the subject.

In other embodiments, the detecting step includes contacting the biological sample with a capture antibody, wherein the capture antibody is an anti-mature THP antibody or an anti-precursor THP antibody, incubating the biological sample and the capture antibody to allow for formation of precursor THP-capture antibody complexes, adding a detection antibody following incubation, wherein the detection antibody is an anti-mature THP antibody wherein the capture antibody is an anti-precursor THP antibody, or is an anti-precursor THP antibody wherein the capture antibody is an anti-mature THP antibody, and detecting binding of the detection antibody to formed precursor THP-capture antibody complexes.

Capture and detection antibodies are described in further detail above.

The method for detecting precursor THP levels in the biological sample can be any method known in the art, such as a direct enzyme-linked immunosorbent assay (ELISA), an indirect ELISA, a competitive ELISA, a sandwich ELISA, or an amplified luminescent proximity homogenous assay (AlphaLISA®). Detailed methods for detecting precursor THP levels in a biological sample are provided above.

The biological sample can be a blood serum sample, a blood plasma sample, or a urine sample.

### Kits

Also provided are kits for performing an ELISA or AlphaLISA® to detect precursor THP, the kit includes a polypeptide disclosed herein. The kit can further include a capture antibody and a detection antibody, where both antibodies are capable of binding the polypeptide and precursor THP. The capture antibody can be anti-mature THP antibody or an anti-precursor THP antibody, as described in detail above. When the detection antibody is an anti-mature THP antibody, the capture antibody is an anti-precursor THP antibody. When the detection antibody is an anti-precursor THP antibody, the capture antibody is an anti-mature THP antibody. The kits can also include any other reagent(s) needed for generating a standard curve and/or performing an ELISA or AlphaLISA assay to detect and/or quantify precursor THP in a biological sample.

### Examples

### Materials and Methods

Generation of anti-peptide antibody for detection of precursor THP. Rabbit polyclonal antibody recognizing the EHP domain of human precursor THP was generated by Biomatic (Ontario, Canada). The synthetic peptide corresponding to human precursor THP 591-612 (C-SVIDQSRVLNLGPITRKGVQAT (SEQ ID NO: 3), conjugated to KLH, was used in immunizations and for antigen-affinity purification of the generated polyclonal antibody.

Western blot. Western blots were carried out in accordance with standard procedures. The following primary antibodies were used: sheep anti-hTHP (R&D Biosystems AF5144) pAb and anti-EHP pAb; secondary antibody antibodies used were rabbit anti-sheep IgG-HRP (AP147P; Millipore) and donkey anti-rabbit IgG-HRP (AP182P: Millipore) Bands were detected by enhanced chemiluminescence (Pierce Super Signal West Pico kit, #34087) on ChemiDoc MP imaging system (BioRad, CA).

Native PAGE and Immunoblot. Native polyacrylamide gel electrophoresis and immunoblot were performed using the Invitrogen 4-16% NativePAGE Bis-Tris precast mini-gel and protocol.

ELISA for precursor THP detection. A custom sandwich ELISA for precursor THP detection was developed using mouse anti-THP monoclonal antibody (sc-271022; Santa Cruz Biotechnology, CA) as a capture antibody and rabbit anti-precursor THP polyclonal antibody generated by Biomatic against SEQ ID NO: 3 as the detection antibody. The detection was performed using donkey anti-rabbit IgG-HRP (Millipore EMD AP182P) and 3,3′,5,5′-Tetramethylbenzidine (TMB) Liquid Substrate System as peroxidase substrate (ThermoFisher Scientific, #34028). The assay was performed using Nunc-Immuno™ MaxiSorp™ 96 well solid plates (Sigma-Aldrich, M9410), 5% BSA (ThermoFisher Scientific, #N502) as a blocking solution, PBS/Tween 0.05%/1% BSA as a washing solution, TMB as substrate (ThermoFisher Scientific, #34028) Stop Solution for TMB Substrates (ThermoFisher Scientific, N600) and standard sandwich ELISA protocol. The standard in the ELISA assay was a custom made 37 amino acids long peptide comprised of mouse anti-hTHP mAb epitope (N-terminal) and EHP sequence (C-terminal), as described in detail above. Minimum detectable level was between 100-50 μg/ml.

Tissue sectioning, immunofluorescence staining, and confocal microscopy. Immunofluorescence staining was performed in accordance with standard protocols on 50 μm sections of 4% paraformaldehyde-fixed kidneys from human biopsies, and sectioned using a vibratome. The following antibodies were used to visualize THP: Anti-Human Uromodulin Antibody, antigen affinity-purified polyclonal sheep IgG sheep (R&D Systems, AF5144), and anti-precursor THP rabbit polyclonal antibody. DAPI was used for staining of nuclei. Oregon Green Phalloidin (Molecular Probes, Eugene, OR) was used for staining of F-actin. Confocal microscopy and image acquisition in four separate consecutive channels were performed using an Olympus Fluoview confocal microscope system (Japan).

Immunoprecipitation of serum THP. Normal human serum was purchased from EMD Millipore. Immunoprecipitation of total THP from serum was performed in RIPA buffer using mouse anti-hTHP monoclonal antibody (B-2, Santa Cruz Biotechnology, sc-271022) or normal mouse IgG (Santa Cruz Biotechnology, sc-2343), both conjugated to agarose beads, for >12 hours at 4° C. Binary antigen-antibody complexes were eluted with 4×SDS-PAGE sample buffer (1:2 ratio of beads to buffer), and subjected to electrophoresis followed by Western blotting. Typically, 1 or 10 ml of human serum was used in immunoprecipitations. The serum was initially depleted from IgGs by pre-incubation with high capacity protein A/G agarose beads (ThermoFisher Scientific, #20423).

Epitope mapping of mouse anti-THP mAb. Mouse anti-hTHP monoclonal antibody B-2 (Santa Cruz Biotechnology, sc-271022) had a manufacturer's defined epitope region between residues 291 and 425 of human THP (SEQ ID NO: 1). To identify its true epitope a PepScreen approach was used from Sigma Aldrich. In short, a peptide library of 25 peptides, of 15 amino acids in length each and with 10 overlapping amino acids, was screened in a direct ELISA using biotinylated mouse anti-hTHP mAb and streptavidin-HRP as a reporter. The unique epitope peptide was identified to be THP amino acids 371-385 (of SEQ ID NO: 1; also provided as SEQ ID NO: 5), and this sequence was used to generate a standard polypeptide for precursor THP ELISA.

ELISA for total THP detection. Total THP was measured in samples of human serum and urine using Human UMOD ELISA kit form Sigma-Aldrich (RAB0751).

Statistical analysis. All statistical tests were performed using GraphPad Prism software unless otherwise noted. A two tailed t-test was used to examine the difference in means for continuous data. The Fisher's exact test was used to determine differences between categorical variables. Simple linear regressions were used to determine relationships between two continuous variables. Statistical significance was determined at p<0.05.

Patient population and study protocol. The sample population was obtained from a cohort of hospitalized cirrhotic patients who were non-consecutively enrolled into a study evaluating urea metabolism. Inclusion criteria for the study included a known diagnosis of cirrhosis and age ≥18 years. The diagnosis of cirrhosis was made based on clinical parameters involving laboratory tests, endoscopic or radiologic evidence of cirrhosis, evidence of decompensation (hepatic encephalopathy, ascites, variceal bleeding, jaundice), and liver biopsy if available. Patients were excluded if there was an unclear diagnosis of cirrhosis, if they had prior solid organ transplantation, if they were admitted electively, or if informed consent could not be obtained. For the purposes of this study, we further excluded patients who had active cancer, AKI on admission, hemodialysis at the time of admission, and confirmed pregnancy. Patients who developed AKI were matched for age, gender, baseline kidney function, and severity of cirrhosis (e.g. Model for Endstage Liver Disease2) to patients who did not develop AKI. There were no significant differences between the two groups for demographics, co-morbid conditions, kidney function (baseline and admission), infections, and severity of cirrhosis.

### Studies and Results

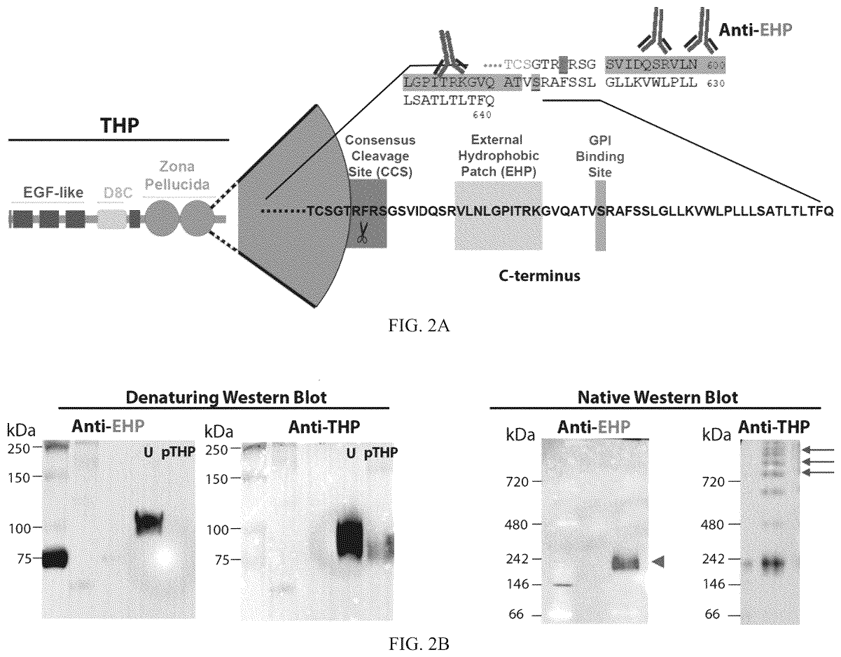

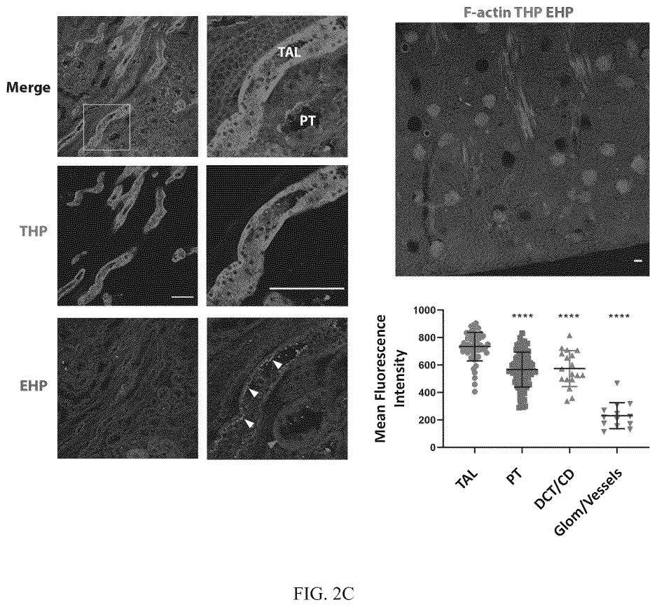

To test the hypothesis that precursor THP could be present in biological samples, an anti-peptide antibody that recognizes the EHP domain of precursor THP (FIG. 1A, top panel; FIG. 2A) was generated and characterized. The presence of precursor THP was then investigated and established in the urine by Western blot (FIG. 2B). Precursor THP had a higher molecular weight (>100 kDa) than urinary or purified mature THP, as expected. Urinary and mature THP were detected using a ship anti-THP polyclonal antibody raised against the mature urinary THP. The specificity of the anti-precursor THP antibody was established by the lack of EHP signal in the purified mature THP (FIG. 2B, left panel). Native immunoblots were performed to determine the polymerization state of precursor THP. Precursor THP was shown to be present as a dimer in its native state, as demonstrated by the discrete, single band at around the 242 kDa marker. Contrastingly, the multimeric native forms of urinary THP presented the characteristic ladder pattern (FIG. 2B, right panel).

The presence of precursor THP in the human kidney was then examined using confocal immunofluorescence. Precursor THP was found to co-localize with mature urinary THP in the thick ascending limbs, where it is maximally expressed at the apical domain (FIG. 1C). This is consistent with release of precursor THP in the urine, as identified herein. However, a less intense but significant precursor THP signal was also detected in other tubular segments, such as proximal tubules (FIG. 1C, right panel). These findings suggest a possible basolateral release and uptake by other tubular segments within the kidney. Without being bound by any particular theory, it is possible that retaining the EHP domain could be an important mechanism by which THP is released into the kidney interstitium, parenchyma, and circulation.

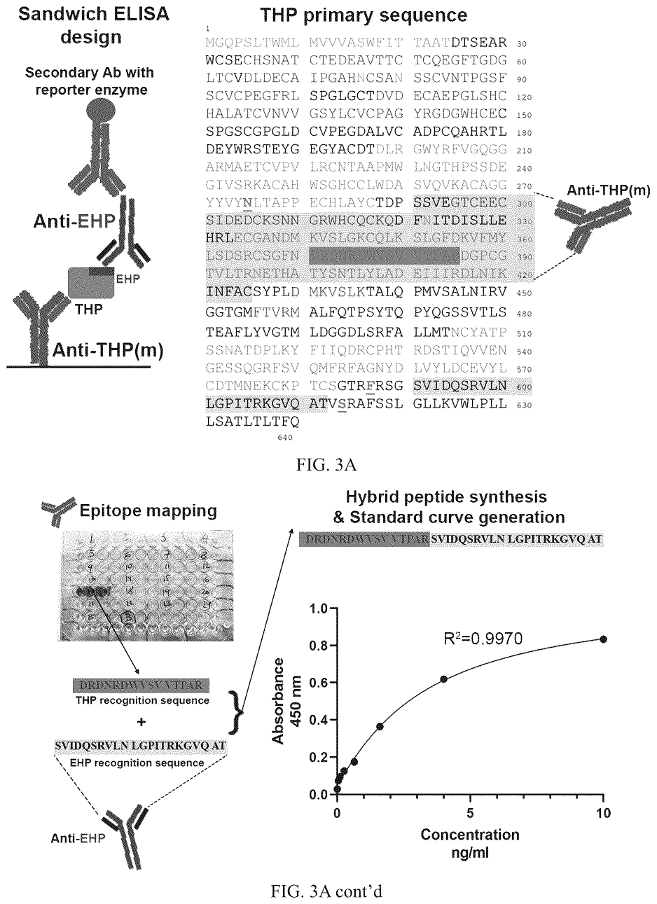

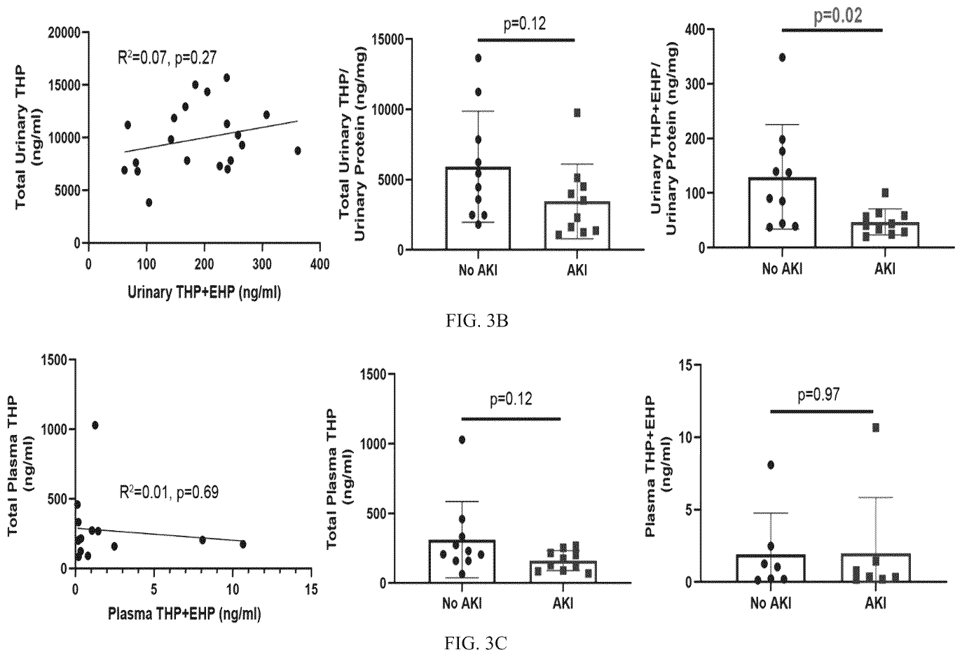

To facilitate the detection and quantitative measurement of precursor THP in the plasma and urine, a specific precursor THP sandwich ELISA was designed and developed. Details of the assay's design are depicted in FIG. 3A. This effort required the generation of a synthetic hybrid calibrator polypeptide: DRDNRDWVSVVTPARSVIDQSRVLNLGPITRKG VQAT (SEQ ID NO: 6; FIG. 3A, right panel). Precursor THP and total THP (assayed using a commercially available ELISA assay) were then measured in the urine and plasma samples collected upon hospital admission from a cohort of 20 patients, half of whom subsequently developed hospital-acquired acute kidney injury (AKI). These patients were part of a previously described study of hospitalized patients with liver cirrhosis, and they were included here entirely a priori, based on a case (AKI)-control design, matching for age, gender, baseline kidney function, and severity of cirrhosis (Table 1). As expected, there were no significant differences between the two groups for demographics, co-morbid conditions, kidney function (baseline and admission), infections, and severity of cirrhosis. FIGS. 3B and 3C show the measurements in the cohort (total and separated by AKI status) in urine and plasma, respectively. Urinary precursor THP was markedly lower and constituted on average only 2.03±0.97% of total urinary THP. There was no significant correlation between precursor THP and total urinary THP (FIG. 3B, left panel), indicating that their expression may be differentially regulated and arguing for a value in their independent measurements. Specifically, precursor THP normalized to total urinary proteins but not total urinary THP was significantly higher in patients who did not develop hospital acquired AKI, indicating that this form of THP is a sensitive indicator of kidney health and its susceptibility to acute injury.

TABLE 1
Demographics and baseline characteristics of cohort of patients with cirrhosis

| | No-AKI | | Development of AKI | | |
|---|---|---|---|---|---|
| | N = 10 | | N = 10 | | P-value |
| Age (s.d) | 56.00 | (7.21) | 57.00 | (9.12) | 0.790 |
| Gender, n (%) male | 6 | (60) | 6 | (60) | 1.00 |
| Race, n (%) white | 8 | (80) | 10 | (100) | 0.474 |
| Etiology of Cirrhosis, n (%) | | | | | |
| HCV | 2 | (20) | 2 | (20) | |
| HCV/ETOH | 2 | (20) | 0 | (0) | 0.539 |
| ETOH | 2 | (20) | 2 | (20) | |
| NASH | 4 | (40) | 5 | (50) | |
| Other | 0 | (0) | 1 | (10) | |
| History of DM, n (%) | 4 | (40) | 4 | (40) | 1.00 |
| History of HTN, n (%) | 5 | (50) | 4 | (40) | 0.470 |
| History of Ascites, n (%) | 6 | (60) | 9 | (90) | 0.303 |
| Infection on Admission, n (%) | 1 | (10) | 4 | (40)* | 0.303 |
| Baseline sCr (s.d) | 0.94 | (0.30) | 1.00 | (0.31) | 0.648 |
| Baseline eGFR (s.d) | 84.60 | (26.73) | 78.10 | (22.27) | 0.562 |
| Admission Heart Rate (s.d) | 85.70 | (13.39) | 92.10 | (11.46) | 0.266 |
| Admission MAP (s.d) | 87.00 | (15.50) | 79.20 | (10.08) | 0.198 |
| Admission WBC (s.d) | 8.10 | (4.90) | 8.27 | (3.88) | 0.932 |
| Admission Hgb (s.d) | 10.85 | (2.28) | 9.90 | (1.47) | 0.284 |
| Admission Platelet Count (s.d) | 106.79 | (67.06) | 98.20 | (32.46) | 0.363 |
| Admission Serum Sodium (s.d) | 137.10 | (3.81) | 131.80 | (7.03) | 0.051 |
| Admission sCr (s.d) | 0.98 | (0.30) | 1.14 | (0.43) | 0.364 |
| Admission eGFR (s.d) | 79.53 | (26.08) | 70.70 | (27.40) | 0.470 |
| Admission Total Bilirubin (s.d) | 3.95 | (4.34) | 3.13 | (2.93) | 0.627 |
| Admission Albumin | 2.95 | (0.69) | 2.56 | (0.57) | 0.186 |
| Admission INR (s.d) | 1.61 | (0.52) | 1.70 | (0.63) | 0.729 |
| Admission MELD score (s.d.) | 15.50 | (6.13) | 17.20 | (5.71) | 0.506 |

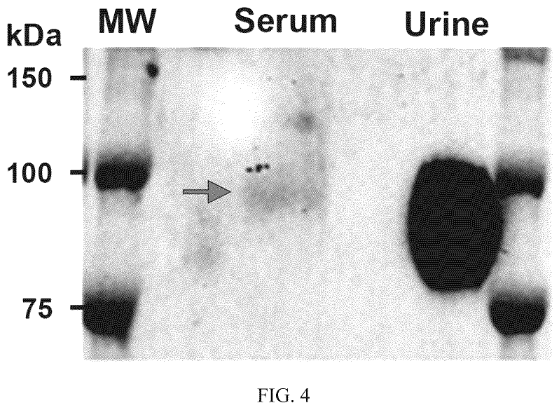

Plasma levels of precursor THP were surprisingly very low, constituting only 0.93±1.94% of total plasma THP. There was no difference in precursor THP on admission in the plasma of patients with or without subsequent AKI, but there was a trend towards higher total plasma THP in those patients who did not develop AKI (p=0.1), which is consistent with the inventor's previous findings. Despite detection of precursor THP within other kidney tubules, suggesting basolateral release by TAL and possible uptake within the kidney, the low levels of precursor THP in the circulation is interesting. Without being bound by any particular theory, the inventors propose two non-exclusive explanations: 1) precursor THP is significantly metabolized/processed within the kidney and scarcely released into the circulation, and that circulating THP is independent of precursor THP; and 2) TAL cells use precursor THP as an important process for basolateral release of THP, but it is further cleaved at the C terminus within the kidney or systemically, making it not easily detectable by anti-precursor THP antibody. The latter proposition can be supported by immunoblotting of immunoprecipitated serum vs. urinary THP, showing that the circulating form has a slightly higher molecular weight (FIG. 4) compared to mature urinary THP (close to 100 kDa, but still lower than precursor THP, which is >100 kDa).

Provided herein is new evidence that the kidney produces a non-polymerizing precursor form of THP by retaining the EHP domain. These findings suggest that this form of THP is independently regulated and provide new insights into the biology of its synthesis and release. Also provided are data that measurements of this non-polymerizing precursor form, using a de novo customized ELISA, provide an independent biomarker in the assessment of risk of kidney injury and chronic kidney disease.

While the disclosed subject matter is amenable to various modifications and alternative forms, specific embodiments are described herein in detail. The intention, however, is not to limit the disclosure to the particular embodiments described. The disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

What is claimed is:

1. A method for predicting a subject's risk for acute kidney injury or risk for chronic kidney disease, comprising:
a. detecting a level of precursor Tamm-Horsfall protein (THP) in a biological sample obtained from the subject; and
b. identifying the subject as having an increased risk of acute kidney injury or risk of chronic kidney disease when the detected level of precursor THP in the biological sample obtained from the subject is lower than mean levels of precursor THP in biological samples from a control population.

2. The method of claim 1, wherein the step of detecting the level of precursor THP comprises:
a. contacting the biological sample obtained from the subject with an anti-precursor THP antibody; and
b. detecting binding of the anti-precursor THP antibody to precursor THP present in the biological sample obtained from the subject.

3. The method of claim 1, wherein the step of detecting the level of precursor THP comprises:
a. contacting the biological sample with a capture antibody, wherein the capture antibody is an anti-mature THP antibody or an anti-precursor THP antibody;
b. incubating the biological sample and the capture antibody to allow for formation of precursor THP-capture antibody complexes;
c. adding a detection antibody following incubation, wherein the detection antibody is an anti-mature THP antibody wherein the capture antibody is an anti-precursor THP antibody, or is an anti-precursor THP antibody wherein the capture antibody is an anti-mature THP antibody; and
d. detecting binding of the detection antibody to formed precursor THP-capture antibody complexes.

4. The method of claim 2 or claim 3, wherein the anti-precursor THP antibody binds to an external hydrophobic patch (EHP) domain of precursor THP.

5. The method of any one of claims 2-4, wherein the anti-precursor THP antibody binds to an amino acid sequence of SEQ ID NO: 3 (SVIDQSRVLNLGPITRKGVQAT).

6. The method of any one of claims 2-5, wherein the anti-precursor THP antibody does not bind mature THP

7. The method of any one of claims 1-6, wherein detecting a level of precursor THP in a biological sample obtained from the subject comprises use of a direct enzyme-linked immunosorbent assay (ELISA), an indirect ELISA, a competitive ELISA, a sandwich ELISA, or an amplified luminescent proximity homogenous assay (AlphaLISA®).

8. The method of any one of claims 1-7, wherein the biological sample is a blood serum or plasma sample, or a urine sample.

9. A polypeptide comprising a first domain comprising an amino acid sequence having at least 95% sequence identity to SVIDQSRVLNLGPITRKGVQAT (SEQ ID NO: 3), and a second domain comprising an epitope of mature Tamm-Horsfall protein (THP), wherein the first domain and the second domain are directly joined together, or are separated by 1 to 100 amino acids.

10. The polypeptide of claim 9, wherein the second domain comprises an amino acid sequence having at least 95% sequence identity to DRDNRDWVSVVTPAR (SEQ ID NO: 5).

11. A polypeptide comprising an amino acid sequence having at least 95% sequence identity to DRDNRDWVSVVTPARSVIDQSRVLNLGPITRKGVQAT (SEQ ID NO: 6).

12. A polypeptide having at least 95% sequence identity to DRDNRDWVSVVTPARSVIDQSRVLNLGPITRKGVQAT (SEQ ID NO: 6)

13. The polypeptide of any one of claims 9-11, further comprising a flexible linker between the first domain and the second domain.

14. The polypeptide of any one of claims 9-13 wherein the at least 95% sequence identity is at least 98% sequence identity.

15. The polypeptide of any one of claims 9-14 wherein the polypeptide is capable of binding to a mature Tamm-Horsfall protein (THP) antibody and an anti-precursor THP antibody.

16. A polynucleotide encoding the polypeptide of any one of claims 9-15.

17. An expression vector comprising the polynucleotide of claim 16.

18. A cell comprising the expression vector of claim 17.

19. A method for generating a standard curve for an immunoassay for detecting precursor Tamm-Horsfall protein (THP) in a biological sample, the method comprising:
a. adding a known concentration of the polypeptide of any one of claims 9-15 to a container comprising a capture antibody, wherein the capture antibody is an anti-THP antibody or an anti-precursor THP external hydrophobic patch (EHP) antibody;
b. incubating the polypeptide and the capture antibody to form polypeptide-capture antibody complexes;
c. adding a detection antibody to the polypeptide-capture antibody complex, wherein the detection antibody is an anti-mature THP antibody wherein the capture antibody is an anti-precursor THP antibody, or is an anti-precursor THP antibody wherein the capture antibody is an anti-mature THP antibody; and
d. detecting binding of the detection antibody to the polypeptide-capture antibody complex.

20. The method of claim 19, further comprising the steps of:
e. repeating steps a. through d. using varying known concentrations of the polypeptide; and
f. generating a standard curve based upon the binding detected between the detection antibody and the polypeptide-capture antibody complex at the varying known concentrations of the polypeptide.

21. The method of claim 19 or claim 20, wherein the anti-precursor THP antibody binds to an amino acid sequence of SEQ ID NO: 3 (SVIDQSRVLNLGPITRKGVQAT).

22. The method of any one of claims 19-21, wherein the capture antibody is affixed within a well of a microplate.

23. The method of any one of claims 19-22, wherein the immunoassay is an enzyme-linked immunosorbent assay (ELISA) or an amplified luminescent proximity homogenous assay (AlphaLISA®).

24. A method for quantifying precursor Tamm-Horsfall protein (THP) in a biological sample, the method comprising:
a. contacting the biological sample with a capture antibody, wherein the capture antibody is an anti-THP antibody or an anti-precursor THP antibody;
b. incubating the biological sample and the capture antibody to allow for formation of precursor THP-capture antibody complexes;
c. adding a detection antibody following incubation, wherein the detection antibody is an anti-THP antibody wherein the capture antibody is an anti-precursor THP antibody, or is an anti-precursor THP antibody wherein the capture antibody is an anti-THP antibody; and
d. detecting binding of the detection antibody to any formed precursor THP-capture antibody complexes.

25. The method of claim 23, wherein the capture antibody is an anti-THP antibody and the detection antibody is an anti-precursor THP antibody.

26. The method of claim 23 or claim 24, further comprising comparing an amount of binding detected between the detection antibody and any formed precursor THP-capture antibody complexes to a standard curve generated in accordance with any one of claims 19-23.

27. The method of any one of claims 24-26, wherein the biological sample is a blood serum or plasma sample, or a urine sample.

28. The method of any one of claims 24-27, further comprising identifying a subject from which the biological sample was obtained as having an increased risk of developing acute kidney injury or chronic kidney disease when precursor THP is detected in the biological sample at a lower level than mean levels of precursor THP detected in biological samples from a control population.

29. A kit for performing an ELISA or an AlphaLISA® to detect precursor THP in a sample, comprising a polypeptide of any one of claims 9-15.

30. The kit of claim 29, wherein the kit further comprises a capture antibody and a detection antibody.

31. The kit of claim 30, wherein the capture antibody is an anti-mature THP antibody or an anti-precursor THP antibody; and wherein the detection antibody is an anti-mature THP antibody when the capture antibody is an anti-precursor THP antibody, or is an anti-precursor THP antibody wherein the capture antibody is an anti-mature THP antibody.

32. The kit of claim any one of claims 29-30, wherein the capture antibody is an anti-mature THP antibody and the detection antibody is an anti-precursor THP antibody.
